(19) United States
(12) Patent Application Publication
(10) Pub. No.: US 2021/0205169 A1
(43) Pub. Date: Jul. 8, 2021

(54) ELECTRO-ACUPUNCTURE DEVICE FOR CONTROLLING NAUSEA

(71) Applicant: ReliefBand Technologies, LLC, Horsham, PA (US)

(72) Inventors: Thomas Schnieder, Beaver Falls, PA (US); Tyler Bryant, Philadelphia, PA (US); Richard Thomas Briganti, Bala Cynwyd, PA (US); David Smail, Greensburg, PA (US)

(21) Appl. No.: 17/143,150

(22) Filed: Jan. 6, 2021

Related U.S. Application Data
(60) Provisional application No. 62/957,406, filed on Jan. 6, 2020.

(51) Int. Cl. A61H 39/00

(52) U.S. Cl. CPC A61H 39/002; A61H 2201/1635; A61H 2201/5048; A61H 2201/5097; A61H 2201/5005; A61H 2201/5043

(57) ABSTRACT

A device for providing noninvasive electrical stimulation of a single acupuncture site for treatment of nausea with capacitor-based voltage regulated circuitry is disclosed. The device is made up of a wristwatch-like housing adapted to be worn on a human wrist. A band attaches the device to the ventral side of the wrist, positioned over the P6 acupuncture point. The device contains a power supply, electrical circuity and at least two electrodes mounted to the inner face of the device, positioned in electrically conductive contact with the mist, which deliver electrostimulation to the P6 point. Within the electrical circuitry is a voltage boost regulator, which limits the output voltage of the device to 30 Volts. This is done to prevent uncomfortable electric shock to the user when the electrodes do not make sufficient contact with the user's skin.

### CROSS-REFERENCE TO RELATED APPLICATIONS

The present invention claims priority to U.S. Provisional Application 67/957,406 filed Jan. 6, 2020.

### BACKGROUND OF THE INVENTION

### 1. Field of the Invention

The methods and devices described below relate to the field of electro-acupuncture and non-invasive stimulation of nerves.

### 2. Description of Related Art

It has long been known that stimulating the P6 acupuncture point is effective in treating or preventing nausea and/or vomiting. To replace the needles used in traditional acupuncture, this stimulation can be provided non-invasively using electrostimulation, sometimes called electro-acupuncture, utilizing electrodes in contact with the skin over the P6 point. The electrostimulation can be delivered continuously or in pulses sufficient to stimulate the nerves of the P6 point.

### BRIEF DESCRITION OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

The present invention is made up of a wristwatch-like housing adapted to be worn on a human wrist. A band attaches the device to the ventral side of the wrist, positioned over the P6 acupuncture point. The device contains a power supply, electrical circuity and at least two electrodes mounted to the inner face of the device, positioned in electrically conductive contact with the wrist, which deliver electrostimulation to the P6 point. Within the electrical circuitry is a voltage boost regulator, which limits the output voltage of the device to 30 Volts, This is done to prevent uncomfortable electric shock to the user when the electrodes do not make sufficient contact with the user's skin.

The foregoing has outlined rather broadly the more pertinent and important features of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present disclosure. It should be realized by those skilled in the art that such equivalent structures do not depart from the spirit and scope of the invention as set forth in the appended claims.

### BRIEF DESCRIPTION OF THE FIGURES

Other features and advantages of the present invention will become apparent when the following detailed description is read in conjunction with the accompanying drawings, in which:

FIG. 1 illustrates various views of the device attached to a band element according to an embodiment of the present invention;

FIG. 2 illustrates various views of the device attached to a band element with an Apple® Watch integration according to an embodiment of the present invention;

FIG. 3 illustrates the ventral housing of the device containing the electrodes according to an embodiment of the present invention;

FIGS. 4 and 5 illustrates the device's internal circuity components according to an embodiment of the present invention;

FIG. 6 is an assembly drawing of the device according to an embodiment of the present invention; and,

FIGS. 7-10 are schematics for the device according to an embodiment of the present invention.

### DETAILED DESCRIPTION OF PREFERRED EMBODIMENTS

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein to specifically provide an electro-acupuncture device for controlling nausea.

We have developed an electro-acupuncture device which has proven effective for the control of nausea and vomiting. The device, marketed under the trademark Relief-Band®, is worn on the wrist like a wristwatch, with a watch-like housing 100, which is positioned on the underside of the wrist. The housing has at least two electrodes 110 on the inside face 108 (the face in contact with the wrist when secured to the wrist) which are separated so that they are not in electrical contact with one another, a battery and circuitry 112A, 112B inside the housing, and control buttons 103 on the outer face 102. A patient suffering from nausea or vomiting (from seasickness, morning sickness, chemotherapy, or anesthesia) can strap the device onto their wrist and turn it on. When turned on, the device emits an electrical stimulation pulse over the P6 acupuncture point (corresponding to the superficial course of the meridian nerve through the wrist). Within several minutes, most patients experience a substantial relief of nausea. The device uses non-invasive nerve stimulation whereby electricity is passed through two electrodes to stimulate nerves located on the ventral side of the wrist (this anatomical position is sometimes referred to as the palmar side of the wrist).

The treatment provided by the device is sometimes referred to as electro-acupuncture, which is a form of acupuncture, and the ventral site of application is referred to in the acupuncture art as the P6 point, pericardium 6 point, or master point of the pericardium meridian (sometimes referred to as the vascular meridian). A primary object of the invention is to provide a non-chemical, non-invasive, painless and inexpensive method of alleviating nausea.

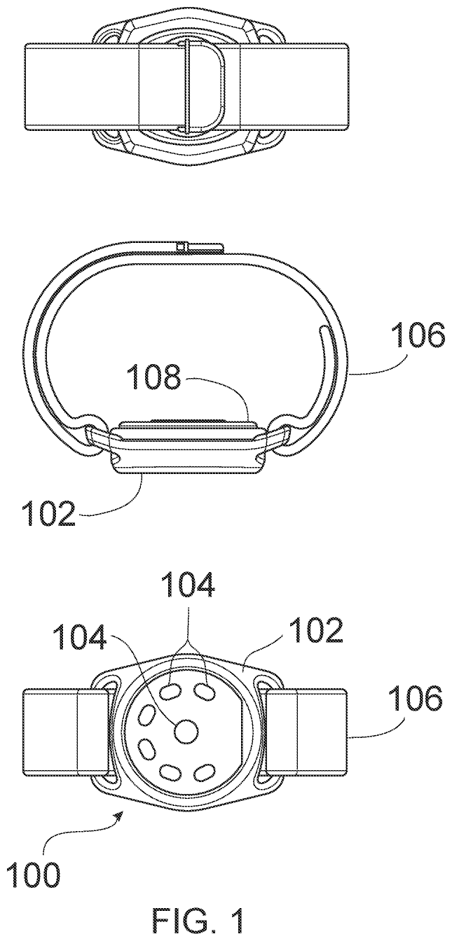

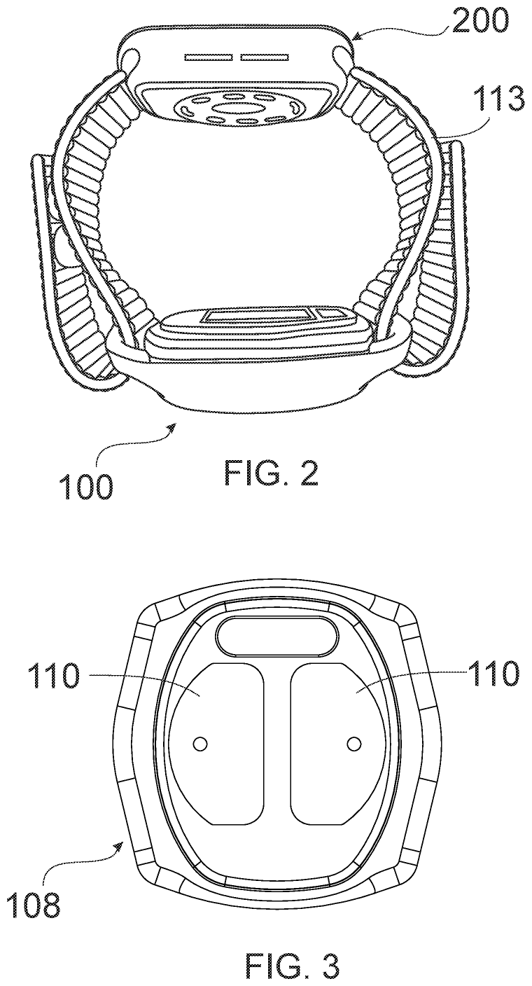

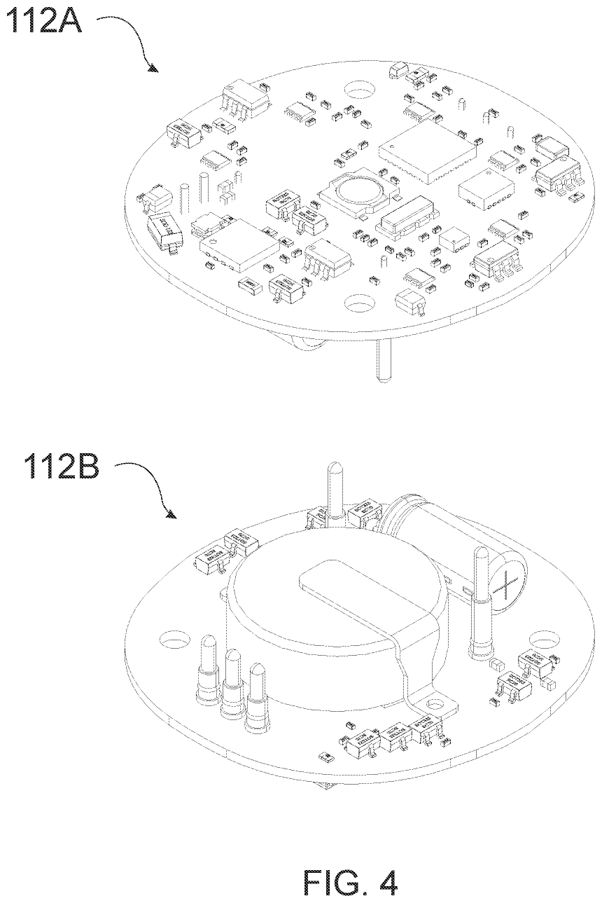

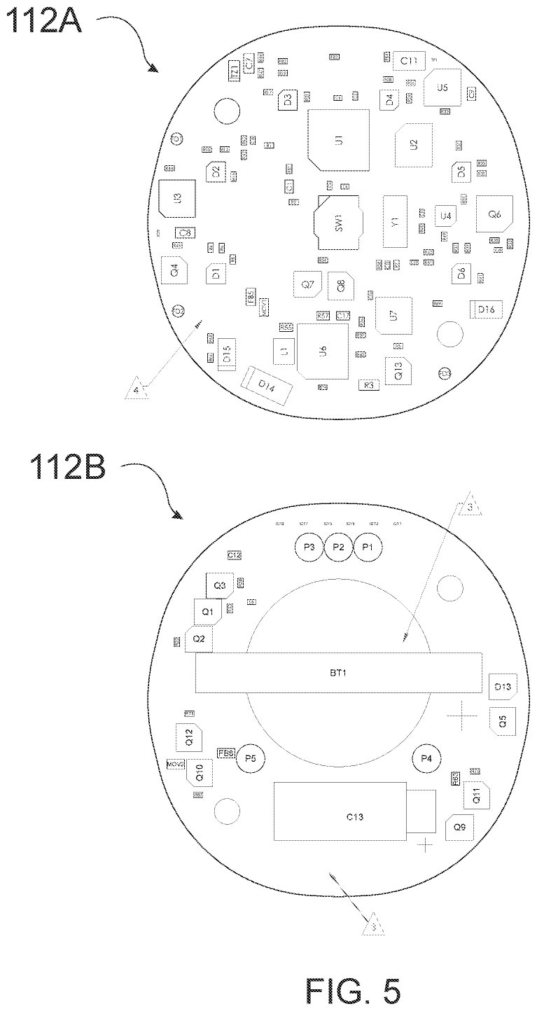

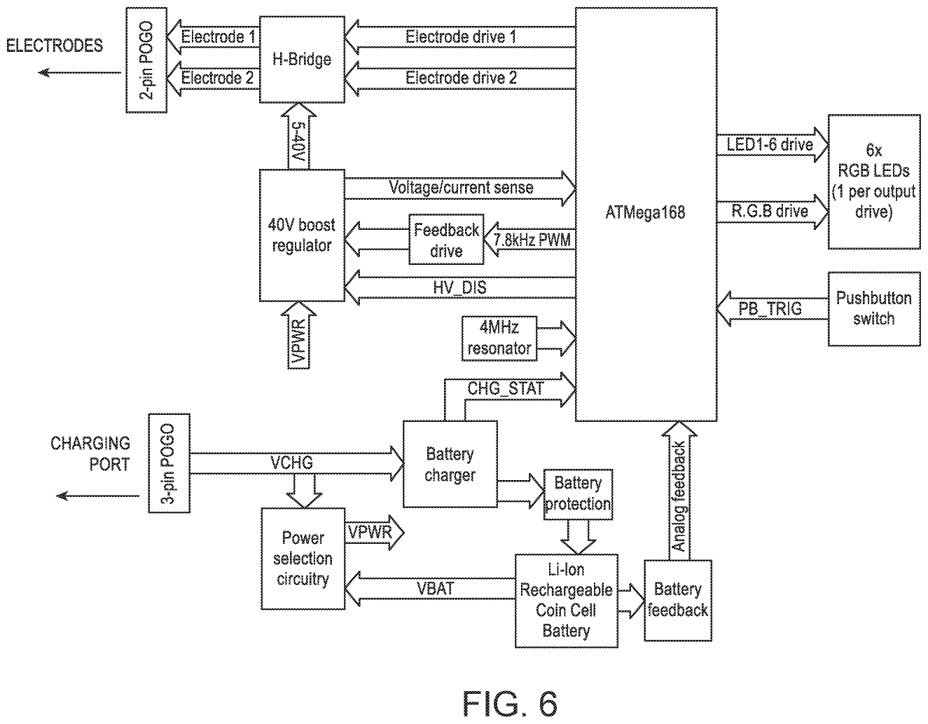

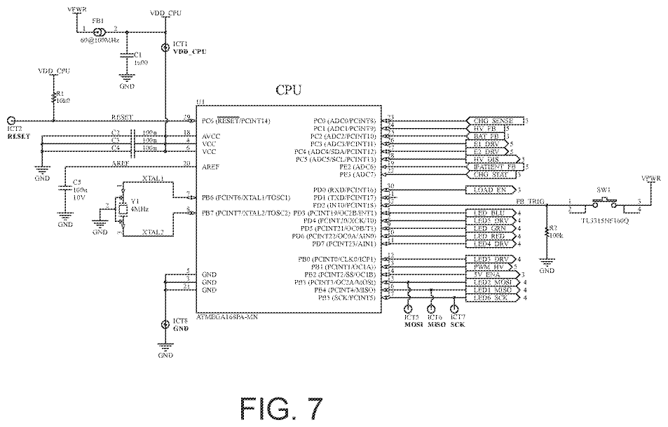

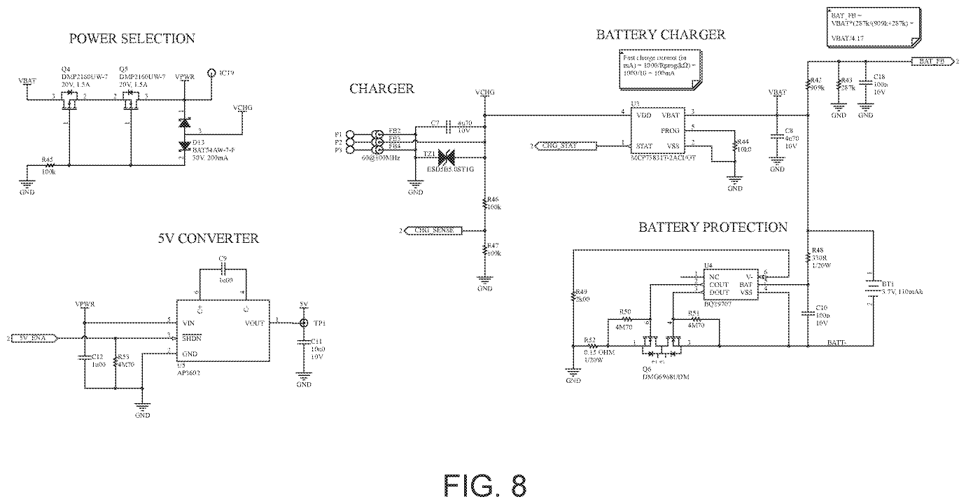

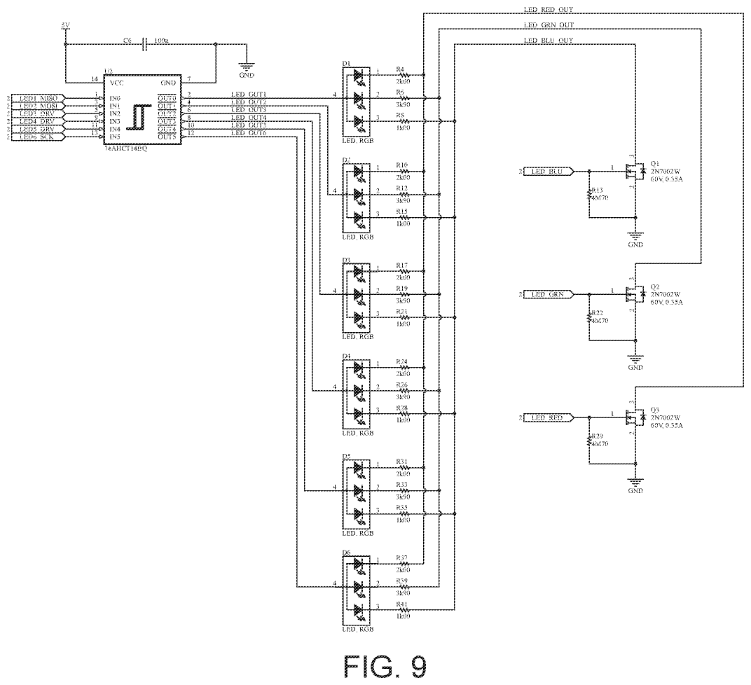

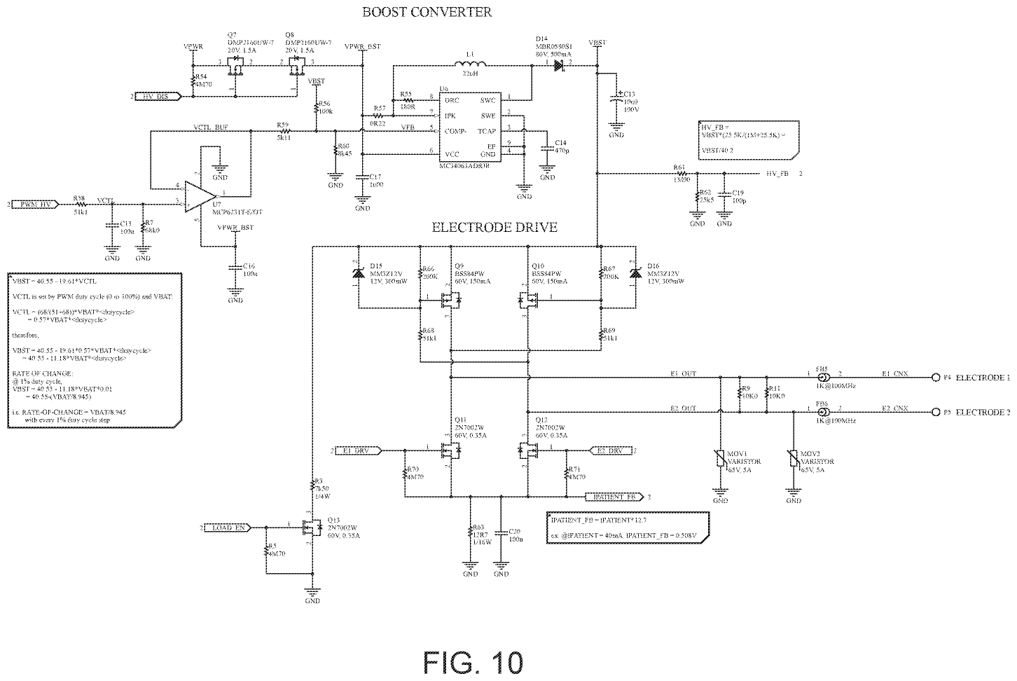

Referring now to any of the accompanying FIGS. 1-10, the present invention provides a non-invasive nerve stimulation device, wherein the electrodes 110 of the device are in electrically conductive contact with skin at acupuncture point P6 of the wrist. Advantageously, the device of the present invention replaces the transformer-based hardware used in traditional nerve stimulation devices with a capacitor-based hardware F.10.

In the process of modeling and testing the updated Capacitive-Drive circuitry, we've discovered an important difference in the two methodologies. The original transformer-drive method was a purely current-driven approach, meaning the output voltage would increase to well over 100V if a light load was present (i.e. poor skin contact). The new approach uses a hybrid voltage/current drive methodology, which limits the voltage to a maximum of 30V under light load conditions. This limit is defined by a voltage boost regulator F.10, which is used instead of the transformer to create a high voltage pulse output. Whereas a transformer-based drive will continually increase output voltage to reach an expected output current level, the transformer-less circuit can only increase voltage up to the maximum output of the boost regulator.

It should be noted that when the device is worn properly, meaning that relative body impedance is in the 500-ohm range as specified in the original v1.5 test document, the two electrode drive circuits operate identically. It is only when the body impedance increases above 750-800 ohms that the differences would he seen (new design clamps output voltage to 30V; old device continues to increase output voltage).

While this new drive technique presents a fundamental difference at light loads compared to the transformer-drive circuit of v1.5 and v2.0, there are several benefits to this approach:

### Safety and User Comfort

Limiting the output voltage to 30V reduces the chance of an uncomfortable shock when adjusting the device or putting it on while it is powered and running. We have currently designed the output waveform to be identical to the output of the transformer for regulatory approval purposes but it would be very easy to modify the waveform to possibly be more comfortable to the user in the administration of treatment.

### Active Feedback

Since the drive circuit is not isolated from the patient through a transformer, we can accurately measure patient load current, so it is possible to alert the patient (by flashing the LEDs 104 in a certain pattern) if the device detects a light load condition caused by poor skin contact.

### Size

The components used to replace the transformer on the new design 112A, 112B occupy approx. 80% of the original transformer circuitry footprint, and all are less than 1 mm tall (current transformer is 5.3 mm tall).

Cost

Transformer replacement circuitry looks to be approx. $0.15-$0.20 cheaper than the transformer and its associated circuitry, in 50k volumes.

### Battery-Saving

In order for the new circuit to output the correct therapy to the patient, the device needs to know if the electrodes are touching the skin. Since the device is able to know if the electrodes are touching a user, we now have a method to turn the device off if not being used after a specific duration. This should significantly increase battery life.

### Smart Device Connectivity

In some embodiments, the device 100 is configured to be worn with a smart watch or other device 200. In some embodiments, circuitry is included to allow the device to connect to smart wearable devices or other devices like smart phones. The device may employ Bluetooth, Wi-Fi, NFC, cellular connection, or other wireless connectivity technologies to accomplish this. The device's function may be facilitated by input from a smart device via an app or other control mechanism, and the device may provide feedback data to the smart device regarding electrical connection quality, stimulation format or pattern, or other information.

### Variations

Although a transformerless design is described, in some embodiments, a transformer is provided. In other embodiments, a transformer with smart load sensing circuit is provided.

### Other Features

Induction-charging Technology is provided to eliminate the need for cables for recharging the device. In other embodiments, a portable battery charger is configured to be used with the device. Although the battery life of the device should be sufficient for multiple sessions, the charger allows the user to dock the device into the charger and provide several cycles of charge for unplanned events and to top-off charge easily when not in use. The charger is sized to fit in the users pocket so it is low-profile. It also has standard charging ports that could charge other devices. Currently, the device uses matching pogo-pin/magnet technology for the interface between the device and the charger but induction charging is also possible. The band element of the device provides extra security to ensure the device remains docked in the charger during use. The band element will be discussed in further detail below.

### Band Element

The device is configured to be used with a band element for securing the device to a wrist of the wearer. In one embodiment, a single-strap band element 106 is provided. This features a unique weave of stretchable strap material that allows a single strap to be weaved through the devices top and bottom openings to secure the device to a wrist (could be an ankle, waist or any location that wraps around a perimeter). Velcro is strategically-placed in locations on the strap to secure to itself and maintain the correct size and optimal tension to ensure the electrodes are in contact with the user.

In another embodiment, a double-strap band element 113 is provided. This type of band-loop technology lends itself nicely to allow our nerve-stimulation devices to become modular. Since the Sport Device was designed to be used with this loop-based technology, it has symmetric top and bottom openings. We now have a method to allow use of any smartwatch on the opposite side of the user's wrist.

Although the invention has been described in considerable detail in language specific to structural features, it is to be understood that the invention defined in the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

We claim:

1. A device to deliver electrostimulation comprising:
a wristwatch-like housing having a size and shape adapted to be worn on a user's wrist, a first face and a second face, a space between the first face and the second face, enclosed to form a cavity;
the first face having at least two electrodes mounted to it opposite the cavity, the electrodes separated relative to one another;
a first opening adjacent to the first face, through which a band may be passed, a second opening adjacent to the first face, opposite to the first opening, through which the band may be passed, the band being passed through the first and second openings and wrapped around the user's wrist to secure the device to the user's wrist, the first face oriented so that the at least two electrodes are in electrically conductive contact with the skin of the user's wrist;
a user interface on the second face, opposite the cavity;
electrical circuitry mounted within the cavity of the housing, the electrical circuitry providing electrical signals, the electrical circuitry incorporating a voltage boost regulator producing an output voltage, the output voltage limited to a maximum of 30 volts, a power supply; and
the circuitry electrically connected to the at least two electrodes and the circuitry electrically connected to the user interface.

2. The device of claim 1, wherein the housing is made of a material selected from the group comprising: plastic, metal, or rubber.

3. The device of claim 1, wherein the band is made of a material selected from the group comprising: metal, nylon, leather, rubber, plastic, canvas, fabric, wool, woven fiber, or elastic.

4. The device of claim 1, wherein the band is integral to a wristwatch, smart watch, or other device worn on the user's wrist.

5. The device of claim 1, wherein the power supply is a replaceable battery.

6. The device of claim 1, wherein the power supply is a rechargeable battery.

7. The device of claim 6, further comprising a means for charging the rechargeable battery inductively, electrically connected to the rechargeable battery.

8. The device of claim 1, wherein the user interface includes one or more LEDs.

9. The device of claim 1, wherein the user interface includes one or more buttons.

10. The device of claim 1, further comprising a means for wirelessly connecting to a secondary device to control or interface with the device of claim 1.

11. A method of delivering electrostimulation to a user comprising;
positioning at least two electrodes in electrically conductive contact with the skin of a user's wrist;
providing a voltage boost regulator configured to limit the output voltage of the at least two electrodes to a maximum of 30 Volts, and configured to sense the quality of the electrical connection between the at least two electrodes and the skin of the user's wrist, providing a user interface to alert the user if a poor connection condition exists;
sensing a poor electrical connection between the at least two electrodes and the skin of the user's wrist;
limiting the output voltage of the electrodes to a maximum of 30 Volts;
alerting the user to the poor electrical connection;
correcting the poor electrical connection; and
delivering electrostimulation to the user.

12. The method of claim 11, further comprising a method of continuously or periodically sensing the quality of the electrical connection between the at least two electrodes and the user's wrist and alerting the user.

13. The method of claim 11, further comprising a method of continuously or periodically sensing the absence of an electrical connection between the at least two electrodes and the user's wrist, and cutting power to the electrodes.

14. The method of claim 11, wherein the device will not deliver electrostimulation until a quality electrical connection between the at least two electrodes and the user's wrist is established.

15. The method of claim 11, wherein the user is alerted using a visual signal.

16. The method of claim 11, wherein the user is alerted using an audible signal.

17. The method of claim 11, wherein the user is alerted using a signal to a secondary device, the secondary device producing an alert either audible, visually, or both.

18. A device to deliver electrostimulation comprising:
a wristwatch-like housing having a size and shape adapted to be worn on a user's wrist, a first face and a second face, a space between the first face and the second face, enclosed to form a cavity;
the first face having at least two electrodes mounted to it opposite the cavity, the electrodes separated relative to one another;
a first opening adjacent to the first face, through which a band may be passed, a second opening adjacent to the first face, opposite to the first opening, through which the band may be passed, the band being passed through the first and second openings and wrapped around the user's wrist to secure the device to the user's wrist, the first face oriented so that the at least two electrodes are in electrically conductive contact with the skin of the user's wrist;
a user interface on the second face, opposite the cavity;
electrical circuitry mounted within the cavity of the housing, the electrical circuitry providing electrical signals, the electrical circuitry incorporating a transformer, a smart load sensing circuit, producing an output voltage, the output voltage limited to a maximum of 30 volts, a power supply; and
the circuitry electrically connected to the at least two electrodes and the circuitry electrically connected to the user interface.
